(19) United States
(12) Patent Application Publication
(10) Pub. No.: US 2008/0140049 A1
(43) Pub. Date: Jun. 12, 2008

(54) Microneedle Device for Transdermal Transport of Fluid

(75) Inventor: Andrew James Kirby, Cyncoed (GB)

Correspondence Address: MERCHANT & GOULD PC, P.O. BOX 2903, MINNEAPOLIS, MN, 55402-0903, US

(73) Assignee: Functional Microsructures Limited, Cardiff (GB)

(21) Appl. No.: 11/793,076

(22) PCT Filed: Dec. 16, 2005
(86) PCT No.: PCT/GB2005/004898

§ 371 Date: Feb. 26, 2008

(30) Foreign Application Priority Data
Dec. 17, 2004 (GB) 0427762.0
Aug. 16, 2005 (GB) PCT/GB05/03224

(51) Int. Cl. A61M 5/28; A61M 5/32

(52) U.S. Cl. 604/506; 604/272; 604/173; 604/257

(57) ABSTRACT

A device (10) provides for transport of material across or into a biological barrier (2), the device includes a puncturing projection (11c) associated with a first portion of a substrate (1), the first portion of the substrate being movable between a first substrate position in which, in use, the puncturing projection is in puncturing contact with the biological barrier so as to form a flow path for said material across or into said biological barrier and a second substrate position in which, in use, the puncturing projection is at least partially retracted from the first position, wherein the substrate is resiliently deformable or displaceable from the first to the second substrate position by a biasing device associated with the substrate.

The invention relates to a device and method for transporting materials across or into biological barriers, in particular but not exclusively for transporting active pharmaceutical compositions across the stratum corneum of human subjects.

Transdermal drug delivery is an important route for pharmaceutical actives, but the outer layer of skin, the 10-20 micrometer thick layer called the stratum corneum, is an effective barrier for many chemical entities. Hence, the number of pharmaceutically active materials that can penetrate into the body through the skin is very limited, and is defined by factors such as polarity, logP, and molecular size. At the same time, many drugs are being synthesized which are unsuitable for oral delivery (for example, due to instability in the gastrointestinal tract, or first pass liver metabolism). Hence, the skin is an attractive, if problematic, route for delivery of these drugs, as well as drugs that act in the skin, but perhaps have systemic side effects.

Several methods have been developed in order to compromise the skin barrier function to allow penetration of drugs into, and analytes out of (for monitoring purposes), the body. These include sonophoresis, iontophoresis and microneedles. U.S. Pat. No. 3,964,482 describes the use of microneedles to assist in the delivery of drugs across the skin. Microneedles puncture the stratum corneum, allowing passage of drug into the subject, but preferably do not induce a pain response because the microneedles do not penetrate to the dermal layer of the skin which is provided with nerve cells. Many microneedle architectures have been developed, including notched, grooved, hollow and porous microneedles, of various sizes from low micrometer to millimetres in length. One key problem is that of providing drug access to the flow paths produced by the needles. This may be solved by using porous or hollow needles, with a reservoir behind the needles, or by coating the needles with drug, or removing the device comprising the needles after making the holes, then applying the drug. Porous and hollow needles suffer from reduced structural integrity, and theoretically, excised tissue can fill the holes in hollow needles, blocking them. Removal of the microneedle device prior to the application of a drug leaves open the possible influx of unwanted agents, including bacteria and viruses to the holes or flow paths produced, which may stay open for some time after removal.

The device and method of the present invention address some of the problems associated with the devices and methods of the prior art.

In accordance with a first aspect of the present invention, there is provided a device for transport of material across or into a biological barrier, the device comprising a puncturing projection associated with a first portion of a substrate, the first portion of the substrate being movable between a first substrate position in which, in use, the puncturing projection is in puncturing contact with the biological barrier so as to form a flow path for said material across or into said biological barrier and a second substrate position in which, in use, the puncturing projection is at least partially retracted from said first position, wherein the substrate is displaceable from the first to the second substrate position by a fluid disposed between said substrate and said biological barrier.

There may be at least one, and preferably more than one, puncturing projection associated with the first portion of the substrate. On movement to the second substrate position, at least one, preferably more than one, more preferably the majority, and further more preferably all, of the puncturing projections associated with the first portion of the substrate may be at least partially retracted from the first substrate position.

The substrate and biological barrier may effectively form a cavity or receptacle into which fluid may be dispensed. The substrate and biological barrier may form such a cavity or receptacle on dispensing of the fluid between the substrate and the biological barrier. This provides a convenient and simple device for transporting a material (such as a drug) across or into a biological barrier (such as the stratum corneum). The material may be contained in the fluid, or the fluid may act as a transport medium. In this case, the material may pass through the fluid from a reservoir. If the fluid acts as a transport medium, then the substrate may be permeable to the material to be transferred into or through the biological layer. In this way, the device may be arranged so that material may move through the substrate into the fluid.

The term “disposed between said substrate and said biological barrier” includes a situation where the device comprises a cover that is, in use, placed between the substrate and the biological barrier, and where the substrate is displaceable from the first to the second substrate position by a fluid disposed between the substrate and the cover. Those skilled in the art will realize that the fluid is still disposed between the substrate and the biological barrier, merely that there is an intervening cover. In this case, a cavity or receptacle may be formed between the cover and the substrate into which fluid may be dispensed. Additionally or alternatively, a cavity may be formed on dispensing of the fluid between the cover and the substrate.

In use, the fluid may or may not occupy the whole of any space between the substrate and the biological barrier.

Preferably, the device includes said fluid. It is further preferred that said fluid comprises or contains the material to be transported across or into the biological layer. This provides a convenient way of introducing material into a subject.

It is preferred that the device comprises a plurality of puncturing projections. It is further preferred that the puncturing projections are arranged in a regular or pseudoregular array. The plurality of puncturing projections may be integral with, or be attached to, a portion of substantially rigid backing that supports the plurality of puncturing projections. The portion of substantially rigid backing may be attached to the substrate. The portion of the substantially rigid backing is preferably of such a size as to permit a fluid disposed between the substrate and the biological barrier to flex or elastically or plastically deform the substrate so as to move the substrate from the first substrate position to the second substrate position.

It is preferred that the array is arranged across the majority of the substrate. It is preferred that the device is adapted so that a majority (and more preferably substantially all) of the projections may be in the first substrate position at the same time.

The device may be provided with a reservoir for the containment of said fluid. The reservoir may be at least partially filled with said fluid. The device may be provided with a barrier between the reservoir and the first portion of the substrate. The barrier may be effectively removed by dissolution, puncturing, melting or rupture. The device may be provided with more than one reservoir, for example, so as to allow two of more different fluids to be introduced into or through the biological barrier or to mix two components so as to form a mixture or composition to be introduced into or through the biological barrier.

Alternatively and additionally to the provision of a reservoir, the device may be provided with a port for the ingress of fluid to the device. The port may be in communication with the reservoir, if present. The port preferably comprises a valve that resists egress of fluid from the device. A port would allow the replenishment of fluid previously used in a device or would allow the introduction of a new component to a fluid (for example, to produce an unstable reaction product that could not be stored for a long period of time in the device prior to use). For example, the component may be a lyophilized component or a rehydration component. Alternatively, the port could be removable, after use.

The device may be provided with a pathway for providing fluid communication between the first portion of the substrate and one or both of the reservoir and the port, if present.

At least one of the puncturing projections (preferably the majority, and more preferably, all of the puncturing projections) may comprise a tip end for puncturing the biological barrier and a base end, the base end being attached to a first surface of the substrate.

The device may be provided with a cover. The cover may prevent or hinder unwanted or accidental access to the puncturing projections. Alternatively or additionally, the cover may be provided an effective way of attaching the device to the biological barrier. If at least one of the puncturing projections comprises a tip end for puncturing the biological barrier and a base end, the base end being attached to a first surface of the substrate, then it is preferred that the cover faces the first surface of the substrate. The cover may be removable from the remainder of the device. Alternatively or additionally, the cover may be provided with a penetration region associated with each puncturing projection through which the projection penetrates in use. The penetration region may comprise a weaker region compared to the part of the cover not associated with a puncturing projection. The penetration region may be provided by an aperture in the cover. The cover may be arranged so as to resist penetration of puncturing projections other than through a weaker region or an aperture. This may assist in resisting re-entry of a puncturing projection through an aperture. This is especially the case if the substrate is flexible. A portion of substrate provided with one or more puncturing projections may be movable relative to the cover (especially after initial penetration of the biological barrier by the puncturing projections) so that the one or more puncturing projections may not repenetrate the biological barrier. This may involve a movement of the said portion of the substrate to a region that does not contain penetration regions. Alternatively, this may involve a movement of the puncturing projections so that they are at such an angle to the biological barrier that repenetration of the biological barrier is unlikely or, more preferably, not possible. The cover may be provided with an adhesive to adhere the puncturing projections in these non-puncturing orientations or positions.

It is preferred that the cover is provided with an adhesive to aid adhesion of the device to the biological barrier. Furthermore, it is preferred that the cover and substrate are flexible so that the device may be folded. In this manner, the surface of the cover provided with adhesive may be folded against itself. This prevents unwanted reuse or abuse of the device. The cover may be provided with an antibacterial or antiviral material on a surface of the cover that is intended to contact the biological barrier.

The cover may be in sheet form and may comprise a mesh, net or web.

The cover may, in use, be placed between the substrate and the biological barrier.

The cover and substrate may be movable relative to each other so as to form a cavity therebetween. In this case, it is preferred that the device is adapted to allow the introduction of said fluid between the cover and the substrate. This may be achieved, for example, by providing a reservoir or a port for the introduction of fluid between the cover and the substrate. It is further preferred that, in use, the substrate is displaceable from the first to the second substrate position by a fluid disposed between said substrate and said cover.

The device may further comprise an applicator for applying pressure to the substrate so as to urge the puncturing projections into the first substrate position. For example, the applicator may apply pressure or force to a surface of the substrate distal to the surface bearing the at least one puncturing projections. The applicator may be integral with, or separate from, the portion of the device comprising the substrate. The applicator may be a roller or a slide that is movable across the substrate.

The substrate, and the cover if present, may be sheet-like. The substrate may be flexible or elastically-deformable or plastically-deformable by said fluid so that the fluid may displace the substrate from said first substrate position to said second substrate position.

The substrate may comprise a polymeric film, comprising one or more of acrylic, polyurethane, silicone polymers or other polymeric material. The puncturing projections may be deposited onto the substrate or may have been shaped from the substrate.

It is preferred that the fluid is movable by the application of pressure to the device. It is preferred that movement of fluid towards or into the region between the first portion of the substrate and the biological barrier may urge the substrate into the second substrate position. In this case, it is preferred that fluid is moved from another region between the substrate and the biological barrier towards or into the region between the first portion of the substrate and the biological barrier. The applicator, if present, may be used to move the fluid into said space. As an alternative to the applicator, the device may be provided with a pump or other mechanical means for applying localized pressure to the device in order to move the fluid towards the first region of the substrate.

In a preferred embodiment, the volume and/or characteristics of the fluid provided with or in the device may be chosen such that, in use, fluid does not contact the whole surface of the substrate that is provided with puncturing projections at any given point in time. For example, if the device comprises an array of puncturing projections that, in use, have been urged into the first substrate position (i.e. puncturing the biological barrier), then, in use, fluid may only be associated with a proportion of the substrate, thus displacing only those puncturing projections associated with the proportion of the substrate into the second substrate position. The remainder of the puncturing projections (i.e. those not associated with said proportion of the substrate) remain in the first substrate position i.e. puncturing the biological barrier. The fluid may act like a droplet under the surface of the substrate that can be pushed to different regions of the space between the substrate and the biological barrier. As pressure is applied on a part of the surface near to the fluid, the projections associated with proportion of the substrate formerly in the second substrate position are pushed back into the skin, whilst at the same time, and by the same process, the fluid “bubble” is moved to a different part of the space between the substrate and the biological barrier, moving the substrate to the second substrate position and facilitating drug flux through the skin in that area. This process can be repeated indefinitely. The surface of the substrate provided with the at least one puncturing projection may be adapted so that the fluid may form a droplet-like shape between said surface and the biological barrier. Said surface may be coated with a low-energy coating to achieve this.

The substrate may be semi-permeable.

In another embodiment, a foam formulation can be used as the fluid. Alternatively, or in addition, the fluid may form a porous solid in response to a stimulus, such as heat, moisture, light or movement, or the mixing of two or more elements together.

The fluid (or resultant porous solid as described above) can also fulfil the role of keeping the perforations in the biological barrier patent for longer. This allows longer term administration of chemical or biological entities across the biological barrier. The force needed to puncture the skin, for example, is considerably greater than that needed to keep the hole in the skin open once punctured. The fluid can act as a method of keeping the holes open and its ability to do this will depend on various factors, including rheology, viscosity, pressure applied, and whether any solid is formed from the fluid.

Movement of the substrate from the first substrate position to the second substrate position may be affected by changing a property of the fluid, such as its total volume, flow characteristics or local shape and volume in the vicinity of a puncturing projection. The properties of the fluid may be changed by one or more of temperature, electromagnetic radiation, electricity, magnetism, humidity, motion, pressure or by chemical stimulus.

At least one (and preferably more than one, more preferably a majority and even more preferably substantially all) of the puncturing projections comprises a microneedle. It is preferred that each of the puncturing projections is 10 microns to 2 mm long, more preferably greater than 100 microns long and further more preferably less than 1000 microns long. It is preferred that the length of the puncturing projections is about 200 to 500 microns, more preferably 200 to 400 microns. Such a length of projection minimizes the chance of a projection reaching the dermis which is provided with nerves which generate pain response. The preferred length may vary depending on the biological layer which is intended to be punctured by the projections.

For example, mucosal layers may require projections of different length than the stratum corneum, whilst stratum corneum thickness varies considerably at different anatomical sites. Furthermore, the length of the needles will reflect the need to overcome the inherent flexibility of the skin, and the need to pass through layers (for instance a skin contact layer) in the device.

Each puncturing projection may be cylindrical (for example, right circular cylindrical), conical or pyramidal in shape. Each projection may be hollow or porous, but it is preferred that each projection is solid i.e. substantially non-porous and not provided with a cavity therein. Solid projections are stronger and easier to fabricate.

At least part of the outer surface of one or more of the puncturing projections may comprise a low friction material. This may be achieved by manufacturing the projection from a low friction material or providing the puncturing projection with a coating or layer of low friction material, Representative examples of such coatings include fluorinated polymers, including organic and silicone polymers such as polytetrafluoroethylene, silicone polymers including polydimethylsiloxanes and fluorosilicones. Alternatively, a lubricant may be applied to the surface of the biological barrier. Such a layer or coating may be provided during manufacture or immediately prior to, or during, use. Such coatings or layers may be permanent or non-permanent.

The device may be in the form of a patch.

The fluid may comprise elements or agents that induce physiological or immunological changes in the organism associated with the biological barrier, such elements or agents including drugs (including topically and systemically acting drugs), a live or attenuated micro-organism (such as bacteria or viruses), nucleic acids, mutagens, toxins, antimicrobial agents or antiviral agents (such as silver).

The fluid is preferably flowable and may be in the form of a gel, ointment, emulsion, cream, solution, suspension or a thixotropic material. The fluid is preferably liquid or has liquid properties.

The fluid may comprise a barrier agent that acts as a barrier when occupying the flow paths produced by puncturing projections to resist unwanted ingress of foreign materials into said flow paths. The fluid may comprise film-forming agents that encourage the formation of a film over the biological barrier once the device has been removed. Examples of such agents include polydimethylsiloxanes (such as dimethicone) (Dow Corning), the VS and SA range of silicone-acrylate polymers as supplied by 3M, nitrocellulose or other polymers. A film may be formed by evaporation of a solvent to leave the polymer deposited in situ.

The fluid may comprise an antimicrobial, antibacterial or antiviral component. Additionally or alternatively, the fluid may comprise a component that changes the properties of the biological barrier, for example, to penetration.

The device may be provided with a control means for controlling the interaction of the device with the biological barrier.

The device is preferably provided with an adhesive to adhere the device to the biological barrier. The adhesive may be provided on the substrate or on the cover, if present. The adhesive may be in the form of a layer. The adhesive may form a seal which is substantially impermeable to said fluid when the device is placed on the biological barrier. In this manner, the adhesive layer may be used to form and define a pathway into which the fluid may flow.

The adhesive layer may be formed around the periphery of the substrate. Alternatively or additionally the adhesive layer may form a serpentine path along the substrate, or may be in the form of a plurality of dots or islands

The adhesive layer is intended to form a bond with the biological barrier of sufficient adhesive strength so as not to be removed accidentally but so as to be removable with a reasonable amount of force. Such a reasonable amount of force should not cause too much damage to the biological barrier or pain or discomfort to the wearer of the device.

The adhesive may be used to create channels to direct fluid in a particular direction, or hold the fluid in a particular position. The adhesive may also be used to hold or separate different fluids, or reservoirs of the same fluid.

The adhesive may have an antimicrobial and/or antiviral activity, either inherently, or due to chemicals incorporated on the surface or within the adhesive. The adhesive can also be made in such a way as to remove or immobilise microorganisms and other noxious agents on the biological barrier during the process of removal of the adhesive element from the biological barrier, for instance during application of the fluid.

In accordance with a second aspect of the present invention, there is further provided a device for transport of fluid-associated material across or into a biological barrier, the device comprising at least one puncturing projection associated with a first portion of a substrate and a means of introducing fluid to the first portion of the substrate, wherein the substrate is moveable between a first substrate position in which, in use, the puncturing projection is in puncturing contact with the biological barrier so as to form a flow path for said fluid associated material across or into said biological barrier and a second substrate position in which, in use, the puncturing projection is at least partially retracted from said first position, wherein, in use, the substrate is displaceable from the first to the second substrate position by said fluid.

The device of the second aspect of the present invention may have the features described above with reference to the device of the first aspect of the present invention. For example, the device of the second aspect of the present invention may comprise a plurality of puncturing projections.

In accordance with a third aspect of the present invention there is provided a method of transport of material across or into a biological barrier, the method comprising:

(i) providing a device comprising a puncturing projection associated with a first portion of a substrate
(ii) providing a fluid
(iii) placing the device against the biological barrier
(iv) causing the puncturing projection to puncture the biological barrier to form a flow path for the transport of material across or into the biological barrier
(v) introducing said fluid between said first substrate portion and said biological barrier
(vi) withdrawing the puncturing projection at least partially from the biological barrier without removing the device from the biological barrier

This method allows the introduction of material into a subject and allows the withdrawal of samples from a subject.

The puncturing projection may be withdrawn fully from the biological barrier. It is preferred that said material is carried by said fluid.

More than one puncturing projection may be associated with the first substrate portion.

The fluid may be flowable. The fluid may be a liquid or have liquid properties.

It is preferred that the introduction of fluid between the first substrate portion and the biological barrier causes the withdrawal of the puncturing projection from the biological barrier. In this case, it is preferred that fluid is moved from another region between the substrate and the biological barrier towards or into the region between the first portion of the substrate and the biological barrier. This may be readily effected by using a substrate that is flexible or elastically or plastically deformable by the introduction of said fluid between the first substrate portion and the biological barrier.

The step of withdrawing the puncturing projection may be performed immediately after causing the puncturing projection to puncture the biological barrier or alternatively, the puncturing projection may be removed from the biological barrier a predetermined time after puncturing the biological barrier. This predetermined time may depend largely on the material to be administered using the device, but may for example be between 10 and 60 minutes.

The method may further comprise the step of causing the puncturing projection to re-enter the entry pathway subsequent to step (v) or (vi). This may help to keep open the entry pathway once the puncturing projection is removed from the biological barrier and thus help to maintain flux of material through or into the biological barrier. This re-entry may act to urge fluid through the flow path formed by the puncturing projection.

The step of puncturing the biological barrier may be repeated. This gives multiple punctures from one projection, thus increasing the flux of material into or through the biological barrier.

The step of attaching the device to the biological barrier may comprise forming a cavity between the substrate and the biological barrier.

The introduction of fluid into a region of the cavity between the substrate and the biological barrier associated with the first region of the substrate may be achieved, for example, by applying mechanical pressure to a surface of the substrate distal from the surface carrying the puncturing projection. Alternatively, a pump may be used to move said fluid. This will also allow the fluid to be introduced into the entry pathway.

The device may comprise a plurality of puncturing projections. It is preferred that the step of causing the puncturing projection to puncture the biological layer comprises causing a plurality of puncturing projections to puncture the biological layer. In this case, the steps of introducing fluid between the first substrate portion and the biological barrier, and withdrawing the puncturing projection may comprise introducing the fluid between the substrate and the biological barrier in a first region, causing the puncturing projections associated with said first region to be at least partially withdrawn from the biological barrier. Puncturing projections not associated with the first region are not withdrawn from the biological barrier. The method may further comprise moving the fluid from the first region to a second region between the substrate and the biological barrier. It is preferred that this causes puncturing projections associated with the second region to be at least partially withdrawn from the biological barrier. It is further preferred that moving fluid from the first to the second region causes puncturing projections associated with the first region to puncture the biological barrier or re-enter the flow paths generated by a previous puncturing step.

The device may be provided with a cover. The step of placing the device against the biological barrier may comprise placing the cover against the biological barrier. The step of introducing said fluid between the first substrate portion and the biological barrier may comprise introducing the fluid between the first substrate portion and the cover.

The method may further provide the step of providing a second barrier to the biological barrier. This step may comprise the application of a barrier cream, ointment, adhesive film or spray. For example, a polymer may be applied by spray to the surface of the biological barrier before application of the microneedles. Alternatively, commercially available wound dressing sprays can be used for this purpose. Alternatively or additionally, an adhesive film, such as a semipermeable wound dressing film, could be placed on the biological barrier surface before application of the microneedles. This film may already have perforations in the surface through which the microneedles will pass, and may also contain elements such as lubricants that will facilitate insertion of the microneedles. The film may be placed on the biological barrier first, followed by the microneedles, or may be applied at the same time, where the adhesive film is already attached in some way to the microneedle support. The advantage of the barrier layer on top of the biological barrier is to reduce possibilities of contamination of the holes produced by the microneedles by agents on the surface of the biological barrier.

Any of the steps of the present method may be controlled automatically. The step of causing the puncturing projections to puncture the biological barrier may be subject to automated mechanical control.

In accordance with a fourth aspect of the present invention there is provided a method of transport of fluid-associated material across or into a biological barrier, the method comprising:

(i) providing a device comprising a puncturing projection associated with a first portion of a substrate
(ii) providing said fluid comprising said material
(iii) placing the device against the biological barrier
(iv) causing the puncturing means to puncture the biological barrier to form a flow path for the entry of said fluid across or into the biological barrier
(v) withdrawing the puncturing projection from the biological barrier without removing the device from the biological barrier
(vi) introducing said fluid into said flow path.

The method of the fourth aspect of the present invention may have the features described above with reference to the method of the third aspect of the present invention. For example, the method of the fourth aspect of the present invention may comprise repeating the step of puncturing the biological barrier.

The methods of the third and fourth aspects of the present invention may use the devices of the first and second aspects of the present invention as described beforehand.

In accordance with a fifth aspect of the present invention there is provided a method for facilitating insertion of microneedles into a biological barrier, said method comprising
(i) providing a device comprising microneedles and
(ii) coating or otherwise generating an outer layer on at least part of the surface of at least one microneedle.

It is preferred that more than one and substantially all of the microneedles are provided with said outer layer. It is further preferred that a tip region of the at least one microneedle is provided with said outer layer. The outer layer may be provided on a bore or pathway provided for the egress of fluid from the microneedle. The outer layer may be provided by a low-surface energy material, such as a polymer, typically silicone or organic polymers. Fluorinated polymers such as polytetrafluoroethylene, or fluorosilicones may be used.

The outer layer may change its surface properties after or during the act of insertion.

In accordance with a sixth aspect of the present invention there is provided a method of transferring a material across or into a biological barrier, then method comprising:
(a) providing a device comprising a plurality of puncturing projections capable of puncturing said biological barrier
(b) causing the puncturing projections to puncture said biological barrier so as to form a plurality of flow paths for transfer of material across or into the biological barrier
(c) removing said puncturing projections at least partially from said biological barrier
(d) supplying a fluid to said plurality of flow paths so as to inhibit the closure of said flow paths.

The step of supplying a fluid to said plurality of flow paths may comprise supplying a fluid under pressure to said flow paths. Additionally or alternatively, this step may comprise providing a fluid to said plurality of flow paths and treating said fluid so as to increase its viscosity. This may mean, for example, heating or cooling the fluid so as to increase its viscosity, or by exposing it to ultra-violet light. Alternatively, the fluid may comprise two or more reagents that react to form a solid, porous solid, or a foam. Alternatively, a foam or porous solid could be formed by action of an external stimulus, such as pressure, heat, light or moisture.

The methods of the third, fourth and sixth aspects of the present invention may further comprise the step of pressurizing said fluid in the region of the flow paths after forming the flow paths. This may be affected by manually applying pressure to a portion of substrate close to or above the flow paths or by use of a pump. This may help to keep the flow paths open and encourage fluid to flow into the flow paths.

In accordance with a seventh aspect of the present invention, there is provided a device for transport of material across or into a biological barrier, the device comprising a puncturing projection associated with a first portion of a substrate, the first portion of the substrate being movable between a first substrate position in which, in use, the puncturing projection is in puncturing contact with the biological barrier so as to form a flow path for said material across or into said biological barrier and a second substrate position in which, in use, the puncturing projection is at least partially retracted from said first position, wherein
(i) the substrate is resiliently deformable or
(ii) the substrate is displaceable from the first to the second substrate position by a biasing means associated with the substrate.

In the second substrate position, the first substrate portion may be fully retracted from the first substrate position.

The substrate may be resiliently deformable and displaceable from the first to the second substrate position by a biasing means associated with the substrate

If the substrate is resiliently deformable, then it may be arranged to urge the first substrate portion from the first substrate position into the second substrate position in the absence of a restraining pressure or force placed on the substrate on, or proximate to, the first substrate portion. In this case, the application of force or pressure to the substrate on, or proximate to, the first substrate portion may retain the first substrate portion in the first substrate position. Release of this force or pressure allows the substrate to return to the second substrate position by virtue of the resiliently deformable nature of the substrate.

Alternatively, the substrate may be arranged to urge the first substrate portion from the first substrate position into the second substrate position on the application of force or pressure to a portion of the substrate remote from the first substrate portion. For example, once the first substrate portion is urged into the first position (i.e. pressing the substrate onto the biological layer) it may remain in the first substrate position until a force or pressure is applied to a portion of substrate remote from the first substrate portion. The force or pressure may be required to be above a certain threshold value in order to cause movement of the first substrate portion into the second substrate position. Urging the first substrate portion into the biological barrier may create an elastic distortion of the substrate elsewhere. Such a distortion may not, in the absence of further stimulus, urge the first substrate portion into the second substrate position. However, applying pressure to a portion of the substrate remote from the first substrate portion (and in particular, a portion of the substrate that is under elastic distortion by virtue of the first substrate portion being in the first substrate position) may cause the first substrate portion to move from the first substrate position to the second substrate position (for example, by increasing the elastic distortion of the substrate to such an extent that it is sufficient to move the first substrate portion into the second substrate position).

In use, displacement of the first portion of the substrate from the first substrate position to the second substrate position may be accompanied by flow of fluid into the space or cavity between the first substrate portion and the biological barrier, and thus into the flow path or flow paths formed by the puncturing projection(s).

It is preferred that the resiliently deformable substrate or biasing means is arranged to resist movement of the first portion of the substrate from the second substrate position to the first substrate position.

It is preferred that the biasing means is, in use, disposed between the substrate and the biological barrier.

The biasing means may comprise a spring (for example, in the form of a leaf spring or a compression spring), sponge, gel or microspheres.

If a leaf spring is used as the biasing means, the first portion of the substrate may be attached to a leaf spring. The leaf spring may, in use, be located between the substrate and the biological barrier or it may be on a rear or reverse surface of the substrate.

Sponge or gel is particularly preferred biasing means because the sponge or gel may be impregnated with fluid to be delivered into a flow path created by the microneedle.

Sponge or gel may be provided with apertures through which, in use, microneedles may pass into the biological barrier.

The device of the seventh aspect of the invention may incorporate those features described above with reference to the first aspect of the invention. For example, the substrate may be a flexible substrate, which may be elastically or plastically deformable.

In accordance with an eighth aspect of the present invention there is provided a kit comprising (i) a device for transport of material across or into a biological barrier, the device comprising a plurality of puncturing projections for puncturing the biological barrier and (ii) a barrier-forming material for placement between the biological barrier and the device. The device is preferably a device in accordance with the first, second or seventh aspects of the present invention. The barrier-forming material may be in the form of a composition that may be topologically applied to the skin of a patient prior to mounting the device onto the said skin. The composition may be a cream or a liquid, for example a liquid that may be applied to the skin of a patient by a swab or a spray dispenser. The kit may further be provided with an applicator for applying the composition, such as a swab or spray dispenser. The barrier-forming material may alternatively be in the form of a sheet of material.

In all of the eight aspects of the present invention, the fluid preferably has liquid properties.

The invention will now be described by way of example only with reference to the following drawings of which:

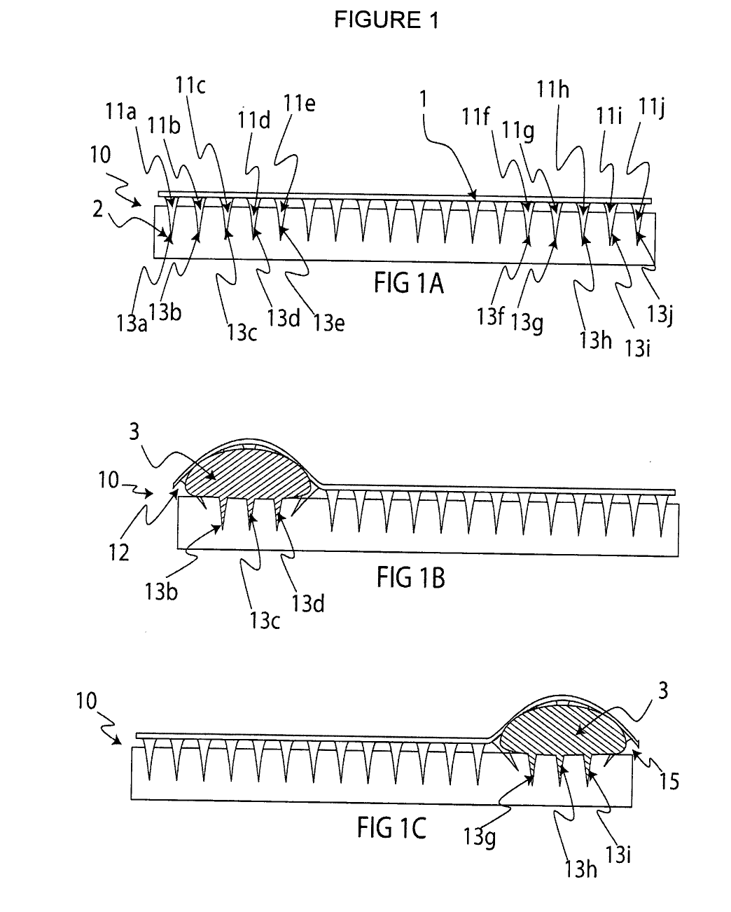

FIGS. 1a-c show a schematic cross-section through a device in accordance with an embodiment of the present invention;

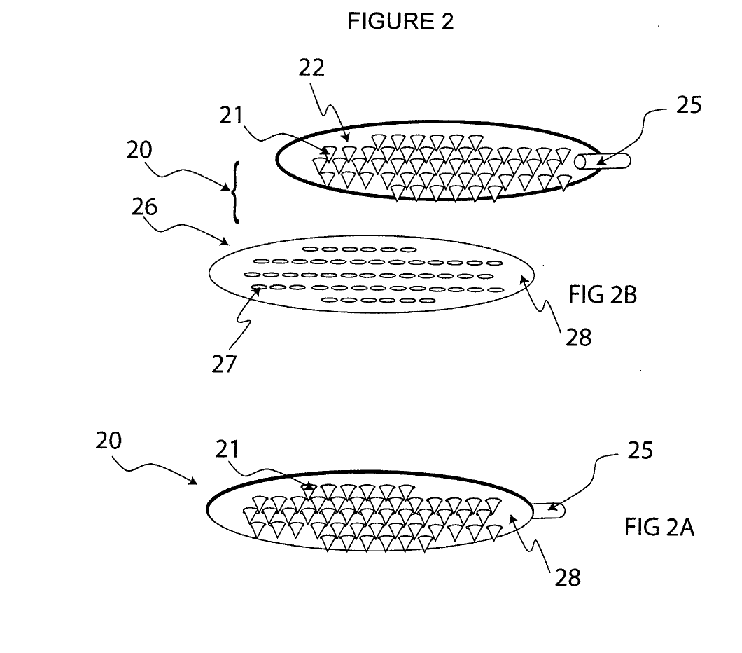

FIG. 2a shows a schematic view of a device in accordance with a second embodiment of the present invention wherein the projections have been deployed though a cover;

FIG. 2b shows an exploded schematic view of the device of FIG. 2a;

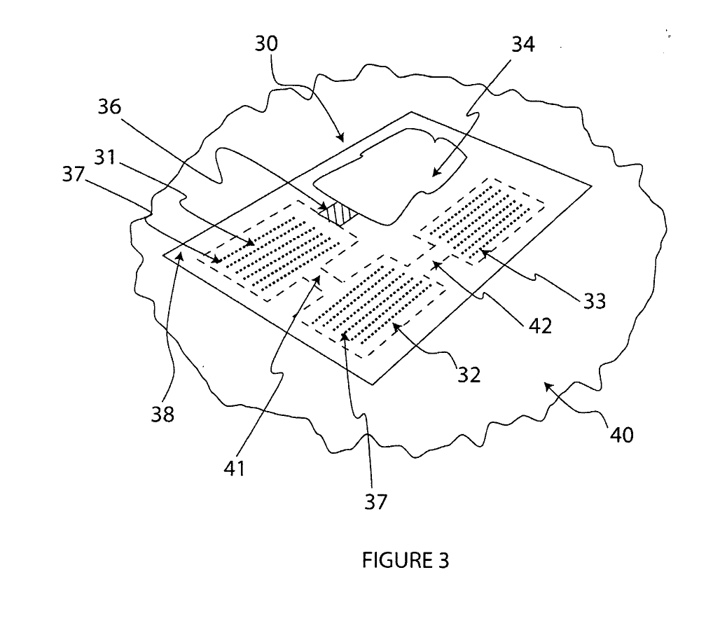

FIG. 3 shows a schematic view of a device in accordance with a third embodiment of the present invention, the device comprising several chambers; and

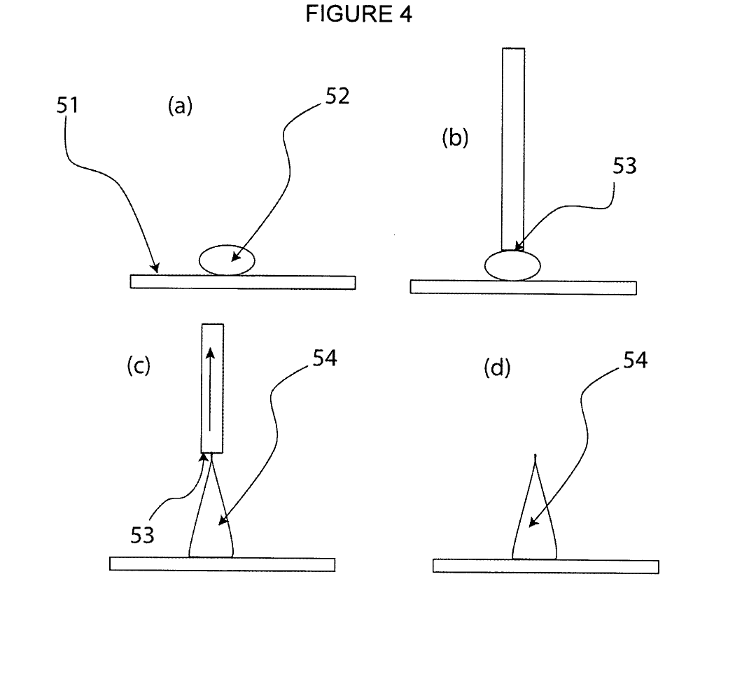

FIG. 4 shows a schematic view of a method of manufacture of a projection that may be used in the device of FIG. 1;

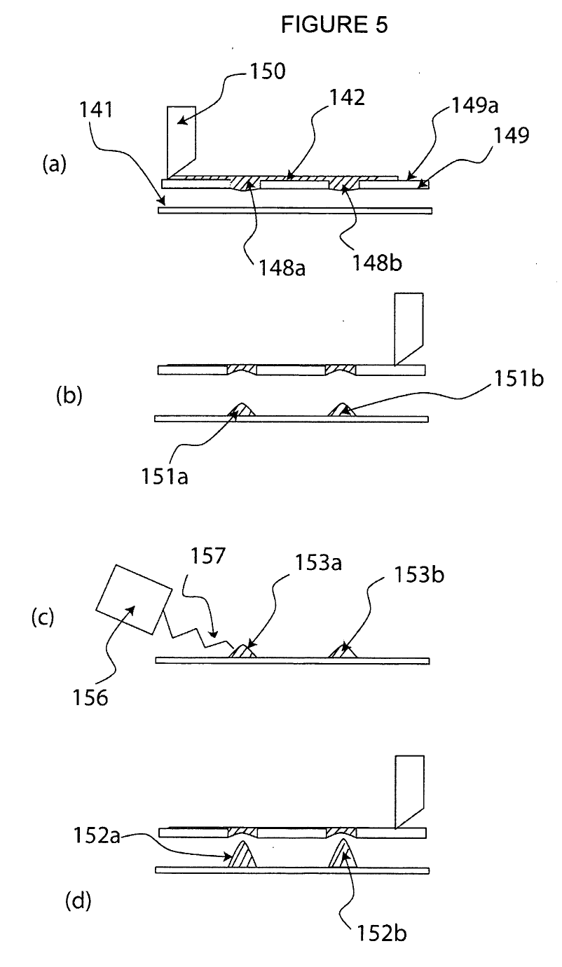

FIG. 5 shows a schematic view of a further method of manufacture of an array of projections that may be used in the device of FIG. 1;

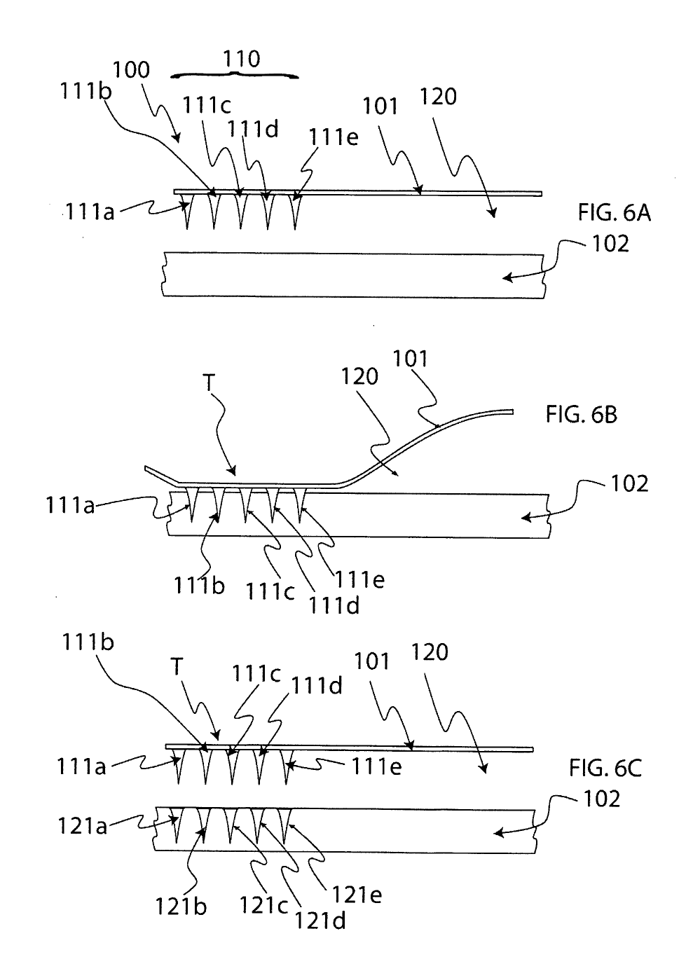

FIGS. 6a-c show a schematic cross-section through a device in accordance with the seventh aspect of the present invention;

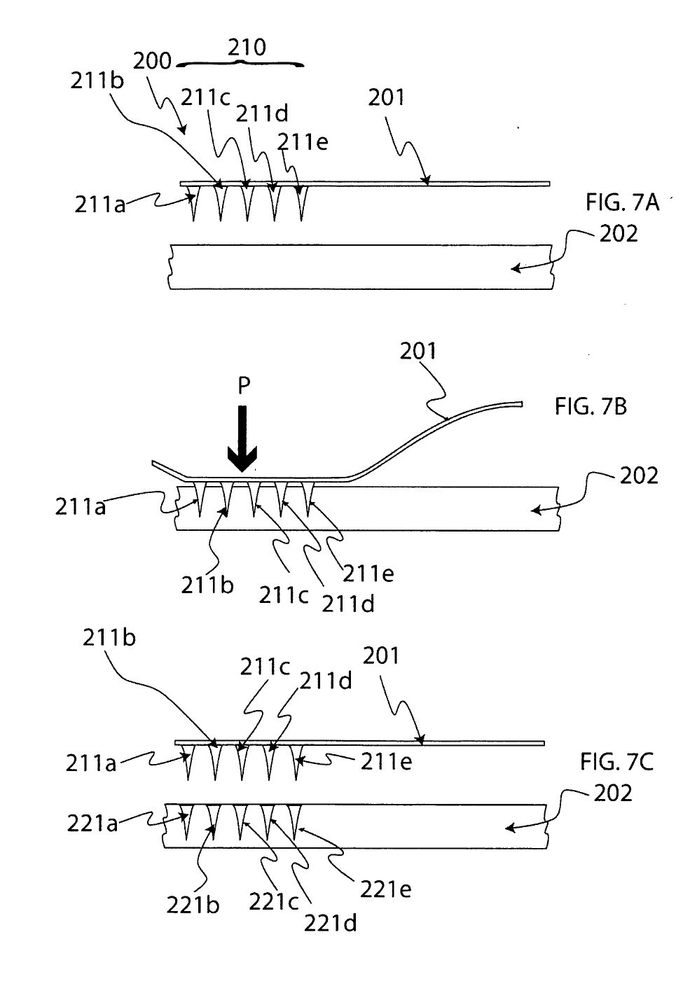

FIGS. 7a-c show a schematic cross-section through an alternative device in accordance with the seventh aspect of the present invention and

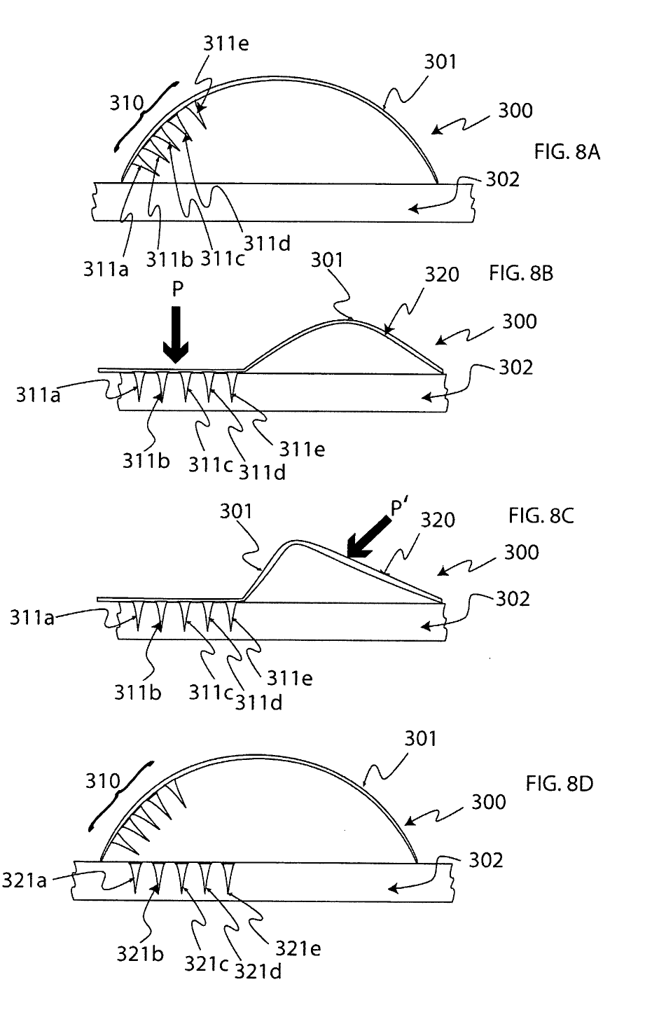

FIGS. 8a-c show a schematic cross-section through a further alternative device in accordance with the seventh aspect of the present invention.

FIGS. 1a-c show a schematic cross-section through a device 10 in accordance with the present invention, the device 10 comprises a plurality of puncturing projections (some of which are labeled 11a-j for convenience) attached to a flexible substrate 1. The substrate is a 30 micron thick sheet of polyurethane. The projections are substantially conical in shape, the base of each cone being associated with the substrate 1. Each projection is about 300 microns long and is formed on the substrate as described now with reference to FIG. 4. To a first solid surface 51, a droplet 52 of liquid is added (for example, silicone resin), preferably by an automated liquid handling means (such as an ink jet, pin contact or screen printer) (FIG. 4a). A second solid surface 53 is brought up to the droplet 52 so that the second solid surface 53 is touching the surface of the droplet 52 (FIG. 4b). The second solid surface 53 is then moved away from the first solid surface 51, drawing the liquid into a needle shape 54 (FIG. 4c). The second solid surface is then removed, leaving needle shape 54 which cures or otherwise solidifies into a solid object (FIG. 4d). This process may be repeated to form many needles, or may be repeated sequentially to build larger needles. In this case, the first solid surface 51 is provided by substrate 1 of FIG. 1, and the needle 54 of FIG. 4 corresponds to the projections 11a-j of FIG. 1.

Alternatively, an array of projections may be formed on the substrate as now described with reference to FIG. 5. A stencil 149 is brought into proximity to a first surface 141 (see FIG. 5a). The first surface 141 is provided by the upper surface of a flexible substrate. The stencil 149 is provided with approximately 1000 apertures, each of 100 μm diameter, in a 1 cm² area. Only two of the apertures, 148a and 148b, are shown here for clarity. A non-solid substance, in this case a UV-curable acrylate 142, was deposited onto the upper surface 149a of stencil 149 using a squeegee 150 which was wiped over upper surface 149a. This wiping action urged the acrylate 142 into the apertures 148a and 148b and urges stencil 149 into contact with the first surface 141. The acrylate 142 adheres to the first surface 141 so that when the stencil 149 is moved away from the first surface 141 (see FIG. 5b), portions 151a, 151b of acrylate remain on the first surface 141. The portions of acrylate 151a, 151b are then cured by exposure to UV radiation (shown schematically as 157) emitted from a UV “spot” source 156 (FIG. 5c) to form solid structures 153a, 153b. The stencil 149 was replaced so that further portions of non-solid substance 142 could be deposited onto the existing structures 153a, 153b. The steps as described with reference to FIGS. 5a, 5b and 5c were further repeated to form an array of needle-like shapes 152a, 152b of approximately 0.7 mm height and having a tip diameter of approximately 20 μm. Needle arrays produced by this method were shown to be capable of penetration of human stratum corneum in vitro. The first solid surface 151 of FIG. 5 corresponds to the substrate 1 of FIG. 1, and the needle-like shapes 152a, 152b of FIG. 5 correspond to the puncturing projections 11a-j of FIG. 1.

In use, the puncturing projections (11a-j) are urged into the biological barrier 2 (in this case the stratum corneum of an adult human) (see FIG. 1a) to form a series of holes (13a-j) or flow paths in the biological barrier 2. Each hole extends through the stratum corneum into the epidermis. Fluid 3 carrying a pharmaceutically active component is then introduced into the device and moved by the application of pressure into the region labeled 12 in FIG. 1b. Movement of the fluid into region 12 causes puncturing projections 11b, 11c and 11d to be moved out of the biological barrier. The fluid 3 enters the holes 13b-d. The active component may enter the bloodstream of the subject via the epidermis and dermis in the region of holes 13b-d, or may act directly on that area in the case of a medication for topical use.

Application of pressure on the external surface of the substrate 1 in the region 12 causes the fluid to move in the cavity defined by the substrate 1 and the biological barrier 2 as shown in FIGS. 1b and 1c. The application of pressure also causes the puncturing projections in the region 12 to either re-enter the holes made by the earlier puncturing process or to make new puncture holes. When the fluid is moved to region 15, the volume and shape of the droplet of fluid, coupled with the flexibility of the substrate 1 cause the puncturing projections 11g-i to be moved out of the biological barrier 2. The fluid 3 then enters the holes 13g-i. The active component may enter the bloodstream of the subject via the epidermis and dermis in the region of holes 13g-i.

In this manner the material may be delivered across or into a biological barrier by merely moving a fluid bearing the material between the flexible substrate and the biological barrier.

FIGS. 2a and 2b show a second embodiment of a device in accordance with the present invention. FIG. 2a shows the device with the projections deployed for penetration of a biological barrier. The device 20 comprises a plurality of puncturing projections 21 arranged in a regular array on the surface of a flexible substrate 22. The substrate is a 30 micron thick sheet of polyurethane. The device further comprises a cover 26 associated with the substrate 22, with the cover 26 and substrate 22 being movable relative to one another so as to be able to form a cavity being between the cover 26 and the substrate 22. The cover 26 is provided with a plurality of weakened regions or holes 27, the puncturing projections, in use, extending through the weakened regions or holes as shown in FIG. 2a. Port 25 is a sealed, injectable port through which fluid may be injected into the cavity between the cover 26 and the substrate 22. In use, the device is pressed against a biological barrier (in this case, the stratum corneum of a human). The surface 28 of the cover 26 in contact with the biological barrier is provided with an adhesive (not shown) that secures the device in place. The surface 28 is also provided with an antimicrobial substance that effectively cleans the surface of the biological barrier in contact with the device. Pressing of the device onto the biological barrier causes the puncturing projections 21 to pass into the biological barrier. Fluid (not shown) is then introduced via port 25 to form a cavity between the substrate 21 and cover 26 that is partially filled with fluid. Pressure may then be manually applied to the device as described above with respect to FIGS. 1a-1c in order to move fluid around the cavity and into the holes or flow paths formed by the puncturing projections.

FIG. 3 shows a third embodiment of a device in accordance with the present invention. The device 30 comprises a flexible substrate 38 that, on mounting the device onto a biological barrier 40, forms three compartments 31-33, each compartment being provided with puncturing projections 37 (shown as a series of dots). The substrate is a 30 micron thick sheet of polyurethane. The device further comprises a reservoir 34 containing fluid (not shown), wherein the reservoir 34 is not provided with any puncturing projections. Adhesive (shown as dashed lines in the Figure) provided on the substrate forms the three compartments 31-33 when the substrate is brought into contact with the biological barrier, the adhesive also forming channels 41 and 42 that extend between compartments 31 and 32, and 32 and 33 respectively.

Application of pressure to the substrate 38 in the region of the compartments 31-33 causes the puncturing projections 37 to penetrate the biological barrier 40. The fluid path between the first compartment 31 and the reservoir 34 is initially blocked by a solid plug 36. Heating of the plug 36 causes it to melt and fluid may then be transferred into the first compartment 31, for example, by the application of pressure to the reservoir 34. The fluid causes the substrate 38 in first compartment 31 to move away from the biological barrier 40 in the region of the fluid (essentially as described with reference to FIGS. 1a-c), thus withdrawing the projections 37 from the biological barrier 40 and allowing the fluid to enter the flow paths generated by the projections. Fluid may be transferred via channels 41 and 42 to second and third compartments 32 and 33 respectively, so that fluid may enter the flow paths generated by the projections associated with those compartments.

The device in accordance with the seventh aspect of the present invention will now be described with reference to FIGS. 6a-c. FIGS. 6a-c show a schematic cross-section through a device in accordance with the seventh aspect of the present invention, the device 100 being mounted for use on a biological barrier 102, in this case the skin of a patient. The device 100 comprises a substrate 101 and a plurality of microneedles 111a-111e associated with a first portion 110 of the substrate 101. The microneedles were formed on the substrate as described above. The substrate is silicone rubber, having a thickness of between 50 and 100 microns. A biasing member in the form of a sponge 120 is situated around the microneedles 111a-e. This may conveniently be achieved by inserting the microneedles into a sponge that is of greater thickness than the length of the micronedles. The sponge 120 is impregnated with fluid (not shown) containing a pharmacologically active ingredient to be delivered to the patient. The use of the device is now described. Referring to FIGS. 6b and 6c, pressure is applied to the top surface (T) of the first portion 110 of substrate 101, compressing the sponge 120 and urging the microneedles 111a-e through the sponge 120 and into the biological barrier 102, thus creating holes or flow paths 121a-e into which fluid may subsequently be dispensed. When pressure is released from the top surface of the first portion of the substrate the sponge 120 returns to its original thickness, urging the microneedles away from the biological barrier 102 (FIG. 6c). Fluid released by compressing the sponge may then pass into the holes or flow paths created by microneedles 111a-e.

A further device in accordance with the seventh aspect of the present invention will now be described with reference to FIGS. 7a-c. FIGS. 7a-c show a schematic cross-section through a device in accordance with the seventh aspect of the present invention, the device 200 being mounted for use on a biological barrier 202, in this case the skin of a patient. The device 200 comprises a substrate 201 and a plurality of microneedles 211a-211e associated with a first portion 210 of the substrate 201. The microneedles were formed on the substrate as described above. The substrate is silicone rubber having a thickness of between 50 and 100 microns.

The application of pressure (P) to the substrate on or proximate to the first substrate portion 210 causes deformation of the substrate so that the microneedles 211a-211e are urged into the biological barrier (see FIG. 7b), creating flow paths 221a-221e. The substrate 201 is resiliently deformable and arranged to urge the first substrate portion 210 from the first substrate position into the second substrate position in the absence of a restraining pressure placed on the substrate on, or proximate to, the first substrate portion. Hence, release of pressure from the substrate permits the substrate to return to its original shape, thus moving the first substrate portion 210 away from the biological barrier.

A further device in accordance with the seventh aspect of the present invention will now be described with reference to FIGS. 8a-d. FIGS. 8a-d show a schematic cross-section through a device in accordance with the seventh aspect of the present invention, the device 300 being mounted for use on a biological barrier 302, in this case the skin of a patient. The device 300 comprises a substrate 301 and a plurality of microneedles 311a-311e associated with a first portion 310 of the substrate 301. The microneedles were formed on the substrate as described above. The substrate is silicone rubber having a thickness of between 50 and 100 microns.

The application of pressure (P) to the substrate on or proximate to the first substrate portion 310 causes deformation of the substrate so that the microneedles 311a-311e are urged into the biological barrier (see FIG. 8b), creating flow paths 321a-321e. The substrate 301 is resiliently deformable but unlike the device of FIGS. 7a-c, removal of the pressure P from the substrate is not accompanied by a movement of the first substrate portion away from the biological barrier; the first substrate portion remains against the biological barrier once pressure P has been removed (see FIG. 8c). In this configuration, a portion 320 of the substrate remote from the first substrate portion 310 is under elastic deformation (see FIGS. 8b and 8c). The application of a force or pressure P′ to portion 320 causes further elastic deformation of the substrate which has the effect of causing the substrate to return (or “snap-back”) to its original shape, therefore moving the first substrate portion into the second substrate position i.e. away from the biological barrier.

Those skilled in the art will realize that the force or pressure P′ may have to be above a certain threshold value to cause the effect mentioned above.

Although the present invention has been discussed primarily with reference to the delivery of materials into or through the stratum corneum, those skilled in the art will realize that the device may be used to deliver materials into or through other biological barriers, such as the blood-brain barrier, mucosal tissue (such as oral, nasal, ocular, vaginal, urethral, gastrointestinal or respiratory), lymphatic vessels or cell membranes. The biological barriers may be in humans or other types of animals, as well as in plants or other organisms, such as bacteria, yeasts or fungi.

The examples given above relate to the administration of a fluid-borne material to a subject via a biological barrier. The device and method of the present invention may be used to withdraw fluid from a subject via a biological barrier, for example for the purposes of conducting tests on said fluid.

1.-45. (canceled)

46. A device for transport of material across or into a biological barrier, the device comprising a puncturing projection associated with a first portion of a substrate, the first portion of the substrate being movable between a first substrate position in which, in use, the puncturing projection is in puncturing contact with the biological barrier so as to form a flow path for said material across or into said biological barrier and a second substrate position in which, in use, the puncturing projection is at least partially retracted from said first position, wherein the substrate is displaceable from the first to the second substrate position by a fluid disposed between said substrate and said biological barrier, wherein, in use, movement of fluid towards or into the region between the first portion of the substrate and the biological barrier from another region between the substrate and the biological barrier urges the substrate into the second substrate position.

47. A device according to claim 46 wherein the fluid is moved by the application of pressure to the device.

48. A device according to claim 46 wherein the fluid comprises a droplet under the surface of the substrate that may be moved to different regions of the space between the substrate and the biological barrier.

49. A device according to claim 46 wherein the substrate is flexible or elastically-deformable or plastically-deformable by said fluid so that the fluid displaces the substrate from said first substrate position to said second substrate position.

50. A device according to claim 46 wherein the volume and/or characteristics of the fluid provided with or in the device are chosen such that, in use, fluid does not contact the whole surface of the substrate that is provided with puncturing projections at any given point in time.

51. A device according to claim 50 comprising an array of puncturing projections, wherein, in use, the fluid is only associated with a proportion of the substrate, thus displacing only those puncturing projections associated with the proportion of the substrate into the second substrate position.

52. A device according to claim 46 wherein there is more than one puncturing projection associated with the first portion of the substrate, and, on movement to the second substrate position, the majority of the puncturing projections associated with the first portion of the substrate are at least partially retracted from the first substrate position.

53. A device according to claim 46 wherein the substrate and biological barrier form a cavity or receptacle into which fluid may be dispensed.

54. A device according to claim 53 wherein the substrate and biological barrier form a cavity or receptacle on dispensing of the fluid between the substrate and the biological barrier.

55. A device according to claim 46, the device comprising a plurality of puncturing projections arranged in a regular or pseudoregular array, the plurality of puncturing projections being integral with, or being attached to, a portion of substantially rigid backing that supports the plurality of puncturing projections, wherein the portion of substantially rigid backing is attached to the substrate.

56. A device according to claim 55 wherein the portion of the substantially rigid backing is of such a size as to permit a fluid disposed between the substrate and the biological barrier to flex or elastically or plastically deform the substrate so as to move the substrate from the first substrate position to the second substrate position.

57. A device according to claim 46 further provided with a reservoir for the containment of said fluid, the device being further provided with a barrier between the reservoir and the first portion of the substrate, wherein the barrier is removable by dissolution, puncturing, melting or rupture.

58. A device according to claim 57, further provided with a port for the ingress of fluid to the device, the port being in communication with the reservoir, wherein the port comprises a valve that resists egress of fluid from the device.

59. A device according to claim 46 further comprising a cover for preventing unwanted or accidental access to the puncturing projections, wherein the cover is provided with a penetration region associated with each puncturing projection through which the projection penetrates in use, wherein the penetration region comprises a weaker region compared to the part of the cover not associated with a puncturing projection or the penetration region is provided by an aperture in the cover.

60. A device according to claim 59 wherein the cover and substrate are movable relative to each other so as to form a cavity therebetween, and the device is adapted to allow the introduction of said fluid between the cover and the substrate, the substrate being displaceable from the first to the second substrate position by the fluid disposed between said substrate and said cover.

61. A device according to claim 46 wherein said fluid comprises or contains the material to be transported across or into the biological layer.

62. A method of transport of material across or into a biological barrier, the method comprising:
(i) providing a device comprising a puncturing projection associated with a first portion of a substrate
(ii) providing a fluid
(iii) placing the device against the biological barrier
(iv) causing the puncturing projection to puncture the biological barrier to form a flow path for the transport of material across or into the biological barrier
(v) introducing said fluid between said first substrate portion and said biological barrier
(vi) withdrawing the puncturing projection at least partially from the biological barrier without removing the device from the biological barrier
wherein the introduction of fluid between the first substrate portion and the biological barrier from another region between the substrate and the biological barrier causes the withdrawal of the puncturing projection from the biological barrier.

63. A device for transport of material across or into a biological barrier, the device comprising a puncturing projection associated with a first portion of a substrate, the first portion of the substrate being movable between a first substrate position in which, in use, the puncturing projection is in puncturing contact with the biological barrier so as to form a flow path for said material across or into said biological barrier and a second substrate position in which, in use, the puncturing projection is at least partially retracted from said first position, wherein
(i) the substrate is resiliently deformable or
(ii) the substrate is displaceable from the first to the second substrate position by a biasing means associated with the substrate.

64. A device according to claim 63 further comprising a fluid, wherein, in use, displacement of the first portion of the substrate from the first substrate position to the second substrate position is accompanied by flow of fluid into a space or cavity between the first substrate portion and the biological barrier, and thus into the flow path or flow paths formed by the puncturing projection(s).

65. A device according to claim 63 wherein the substrate is resiliently deformable, and is arranged to urge the first substrate portion from the first substrate position into the second substrate position in the absence of a restraining pressure or force placed on the substrate on, or proximate to, the first substrate portion.
